(19) United States
(12) Patent Application Publication
(10) Pub. No.: US 2014/0154389 A1
(43) Pub. Date: Jun. 5, 2014

(54) PRODUCTION OF CHEESE WITH S. THERMOPHILUS

(71) Applicant: Lars W. Petersen, Muskego, WI (US)

(72) Inventor: Lars W. Petersen, Muskego, WI (US)

(73) Assignee: DuPont Nutrition BioSciences ApS, Copenhagen (DK)

(21) Appl. No.: 14/112,898

(22) PCT Filed: Apr. 20, 2012
(86) PCT No.: PCT/US2012/034455

§ 371 Date: Jan. 7, 2014

Related U.S. Application Data
(60) Provisional application No. 61/477,211, filed on Apr. 20, 2011.

(30) Foreign Application Priority Data
Jan. 12, 2012 (US) PCT/US2012/021113

(51) Int. Cl. A23C 19/032; A23C 19/072; C12N 1/20; A23C 19/068

(52) U.S. Cl. CPC A23C 19/032; A23C 19/068; A23C 19/072; A23C 19/0688; A23C 19/0684; C12N 1/20
USPC 426/582; 426/61; 426/43; 426/38

(57) ABSTRACT

Methods and compositions for producing cheese with S. thermophilus and a urease inhibitor, and for producing hard or semi-hard cheese, such as cheddar, with S. thermophilus that is partially or completely deficient in its ability to release ammonia from urea are provided. Methods and compositions for reducing, the amount of open texture (e.g., slits, cracks, or fractures) in hard and semi-hard cheeses, as well as hard and semi-hard cheeses that comprise one or more S. thermophilus bacteria that are partially or completely deficient in their ability to release ammonia from urea, are also provided.

This application claims priority to U.S. Provisional Patent Application 61/477,211, filed Apr. 20, 2011, which is hereby incorporated by reference in its entirety, and claims priority to PCT Application PCT/US2012/021113, filed on Jan. 12, 2012, which is hereby incorporated by reference in its entirety.

### BACKGROUND

Streptococcus thermophilus is a thermophilic lactic bacterium used as a lactic ferment in the dairy industry. First used for the manufacture of fermented milks such as yoghurt, it is now increasingly used in cheese production, for example, in production of cheeses that was formerly made with Lactococci bacteria, such a Lactococcus lactis or Lactococcus cremoris.

This bacterium converts lactose in milk into lactic acid, which acidifies the milk. In the case of cheeses, this acidification not only encourages the action of the coagulant and the synaeresis of the curds, but also inhibits the growth of many undesirable bacteria, certain of which are pathogenic bacteria, and allows their elimination at a greater or lesser speed.

The acidifying activity of this bacterium is accompanied by urea hydrolysis activity, which affects the acidification kinetics. Tinson et al (1982) refers to the urea hydrolysis reaction, which converts urea into carbon dioxide and ammonia, resulting in a temporary decrease in the acidification speed, as measured by a pH probe.

On an industrial scale, the hydrolysis of urea by Streptococcus thermophilus poses a number of problems. This is because, in cheese manufacturing for example, the technological operations (cutting of the curds, stirring, etc.) must take place at given values of pH, but in practice these operations are generally carried out at predetermined times. Therefore the variations in acidifying activity due to urea hydrolysis may lead to defects and variability in the texture, moisture level, and ripening properties of the resulting cheeses. Moreover, because ammonia is basic, the production of ammonia increases the time necessary to reach a given pH. This results in the cheese-making equipment being tied up for longer and in an increase of the risk of contamination by undesirable micro-organisms. Furthermore, it is desirable that the cheese-making whey does not contain an excessive amount of ammonia, because this whey is often used as an ingredient in human food and animal feed. The production of ammonia from urea is difficult to control, in part because the urea content of milk is variable (for example, from 2 to 8 mM) and depends in part on the diet of the livestock that produce the milk.

To overcome this problem, Martin et al (1997) proposed measuring the urea content of the milk and then adapting the manufacturing parameters. However, such a system, which requires quantitatively determining the amount of urea, would be highly constraining, and would not resolve the other drawbacks caused by reduction of acidification speed in the presence of urea, such as the equipment being tied up for a longer time, increased risk of contamination, high ammonia content of the whey, etc.

U.S. Pat. No. 6,962,721, which is hereby incorporated by reference in its entirety, refers to the use of Streptococcus thermophilus strains lacking the ability, or having reduced ability, to hydrolyze urea, (herein termed S. thermophilus “ur(−) bacteria”) as lactic ferments in the production of dairy products. U.S. Pat. No. 6,962,721 describes neither how to reduce open texture in hard or semi-hard cheeses, nor hard or semi-hard cheeses with reduced open texture.

### BRIEF DESCRIPTION OF THE FIGURES

FIG. 1 is a graph with a table insert showing exemplary activity profiles of ur(+) and ur(−) bacteria;

FIG. 2 is a graph showing exemplary activity profiles of ur(+) and ur(−) bacteria;

FIG. 3 is a photograph showing an exemplary result from a floating curd experiment in test tubes;

FIG. 4A is a photograph showing floating curd in a vat of milk;

FIG. 4B is a photograph showing a vat of milk without floating curd; and

FIG. 5 is a graph showing an exemplary activity profile of ur(−) and ur(+) S. thermophilus in 2% fat milk.

### SUMMARY

Methods, compositions, and systems for producing cheese with S. thermophilus and a urease inhibitor, and for producing cottage cheese with S. thermophilus that are partially or completely deficient in their ability to release ammonia from urea are provided. Methods, compositions, and systems for reducing the amount of open texture (e.g., slits, cracks, or fractures) in gassy cheeses, which may include cheeses that produce gas during ripening, such as, for example, cheddar cheese, are also provided.

Various exemplary bacterial strains are occasionally referred to herein. Certain strains are referred to by the nomenclature CNCM followed by letters and/or numbers, or DSM followed by letters and/or numbers. These references are the deposit numbers at the Collection Nationale de Cultures de Microorganismes (CNCM) and the Deutsche Sammlung von Mikroorganismen (DSMZ), respectively. All strains referred to by such numbers have been deposited in the respective culture depositories under the reference numbers referred to herein, as follows: CNCM I-2311 was deposited at the CNCM on 14 Sep. 1999 by Texel/Rhodia services and is described in U.S. Pat. No. 6,962,721 which is hereby incorporated by reference it its entirety; CNCM I-2312 was deposited at the CNCM on 14 Sep. 1999 by Texel/Rhodia services and is described in U.S. Pat. No. 6,962,721 which is hereby incorporated by reference in its entirety; CNCM I-2980 was deposited at the CNCM on 26 Feb. 2003 by Rhodia Food SAS, and is described in WO 04/085607 which is hereby incorporated by reference in its entirety; CNCM I-3617 was deposited at the CNCM on 14 Jun. 2006 in the name of Danisco France SAS and is described in WO 08/040,734 which is hereby incorporated by reference in its entirety; DSM 21892 was deposited at the DSMZ on 7 Oct. 2008 in the name of Danisco Deutschland GmbH and is described in WO 10/066,907 which is hereby incorporated by reference in its entirety; and DSM 18344 was deposited at the DSMZ on 14 Jun. 2006 and is described in WO 07/144,770 which is hereby incorporated by reference in its entirety.

In one aspect, methods for producing cheese, such as cottage cheese, are provided comprising the following steps: a) inoculating milk with ur(−) Streptococcus thermophilus bacteria that are not able to release ammonia from urea, or having a diminished ability to release ammonia from urea compared to wild-type S. thermophilus; b) fermenting the milk with the ur(−) Streptococcus thermophilus bacteria; and c) optionally taking further steps, resulting in the final cheese product, which in some aspects is cottage cheese, and in other aspects is a hard or semi-hard cheese, for example, cheddar cheese. In particular aspects, the milk may be inoculated with one or more strains of Lactococcus bacteria, such as, for example and without limitation, Lactococcus cremoris or Lactococcus lactis, in addition to the ur(−) Streptococcus thermophilus (S. thermophilus). See, e.g., methods of making cottage cheese in U.S. Pat. Nos. 6,482,460; 6,238,717; 3,298,836; WO91/00690; and 3,968,256; all of which are hereby incorporated by reference in their entirety.

In certain aspects, the milk includes one or more of cow's milk, goat's milk, sheep's milk, or any other type of suitable milk. In particular aspects, the milk is inoculated with 10⁴ to 10¹³ cfu/ml of ur(−) S. thermophilus, or with 10⁸ to 10¹² cfu/ml of S. ur(−) thermophilus bacteria. In certain aspects, the fermentation time in step b) is from 3 to 7 hours (e.g., 3 hours . . . 4.2 hours . . . 5.5 hours . . . 6.1 hours . . . or 7 hours)

In other aspects, the milk is also inoculated with one or more Lactococcus bacteria, such as Lactococcus lactis or Lactococcus cremoris bacteria. In further aspects, the Lactococcus bacteria are homofermentative Lactococcus bacteria. In certain aspects, the milk is inoculated with 10⁴ to 10¹³ cfu/ml of Lactococcus bacteria or 10⁸ to 10¹² cfu/ml of Lactococcus bacteria.

In particular aspects, the further steps referred in step c) can include, without limitation: i) when pH has reached an appropriate level, for example about 4.65, the coagulum can be cut into cheese curd in order to separate the whey from the cheese curd; and ii) scalding (heating) (e.g., in order to stop the bacterial fermentation process), can be performed, for example, in a cheese vat at the surface of the whey by a steam-injector inserted right below the whey surface and above the cheese curd. In one aspect, the temperature range and times for heating depend on the type of cheese that is being manufactured. As a non-limiting example, cheddar cheese can be produced using an initial temperature of about 88° F. to about 90° F., which can be maintained for example from about 20 to about 25 minutes. After that time, the milk can form curds which can be cut, followed by a heal time of, for example, about 10 to about 15 minutes. The curd can then be cooked at, for example, at about 101° F. to about 103° F. The cook temperature can be maintained at, for example, about 20 to about 40 minutes. The cheese can then be cooled, for example, from about 2.5 to about 2.9 hours. Other temperatures and conditions may also be used for other types of cheeses. For example, cottage cheese can be cooked at a temperature from 126° F. to about 130° F., or higher, which may be a high enough temperature to kill bacteria, such as, for example, S. thermophilus and/or Lactococci bacteria.

Combinations of Lactococci and S. thermophilus may be used in cottage cheese production. This combination may increase the cheese yield. However, the combination may cause cheese curd to float to the top in the vat. The floating curd may make processing the vat difficult. Without wishing to be bound by theory, the presence of floating curd is believed to be due to the presence of urease activity associated with ur(+) S. thermophilus, resulting in release of ammonia and carbon dioxide from urea hydrolysis. Therefore in certain aspects, Streptococcus thermophilus bacteria which are not able (partially or totally) to release ammonia from urea (i.e. the ur(−) S. thermophilus) are used in a process for producing cottage cheese. The above-described floating cheese curd may be eliminated or mitigated by using ur(−) bacteria. In some aspects ur(−) Streptococcus thermophilus bacteria are used in combination with Lactococcus bacteria in a process for producing cottage cheese.

In particular aspects, the ur(−) Streptococcus thermophilus strains are the strains described in U.S. Pat. No. 6,962,721. In some aspects, the Streptococcus thermophilus strains are selected from the group consisting of 298-K (CNCM I-2311), 298-10 (CNCM I-2312), and any mutant or mixture thereof. In particular aspects, ur(−) Streptococcus thermophilus strains are selected from the group consisting of CNCM I-2311, CNCM I-2312, CHCC9908, CHCC4325, and mixtures or mutants, such as ur(−) mutants, of any of these.

In some aspects, a cheese product such as, for example, cottage cheese, cheddar cheese, or any other type of cheese, produced by the methods described herein is provided.

Particular aspects provide the use of a Streptococcus thermophilus ur(−) mutant of a strain selected from the group consisting of CNCM I-2980, DSM21892, CNCM I-3617, CNCM I-3617, CHCC4325, DSM18344, and DSM18111, in a process for producing cottage cheese.

Particular aspects provide methods for producing a fermented milk product such as cheese (e.g., cottage cheese, cheddar cheese, mozzarella, pizza cheese, blue cheese, Swiss cheese, or any other type of cheese), fermented milk, or yogurt comprising: a) contacting milk with Streptococcus thermophilus bacteria and a urease inhibitor; and b) fermenting the milk with the bacteria under conditions such that the fermented milk product (e.g., cheese or yogurt) is produced. In particular aspects, the cheese is cottage cheese or cheddar cheese.

In some aspects, the Streptococcus thermophilus bacteria are able to release ammonia from urea (e.g., strains CNCM I-2980, DSM21892, CNCM I-3617, CHCC4325, and DSM18344). In certain aspects, the Streptococcus thermophilus bacteria are not able to release ammonia from urea or have a diminished capacity to release ammonia from urea compared to wild-type S. thermophilus (e.g., 10% less than wild-type . . . 50% less than wild-type . . . 90% less than wild-type), e.g. CNCM I-2311, CNCM I-2312, CHCC9908. In some aspects, the Streptococcus thermophilus bacteria are a mixture of Streptococcus thermophilus bacteria able to release ammonia from urea and Streptococcus thermophilus bacteria not able to release ammonia from urea or having a diminished capacity to release the same amount of ammonia from urea that is released by wild-type S. thermophilus.

In particular aspects, the urease inhibitor comprises flurofamide. In other aspects, the urease inhibitor comprises a diphenol, a quinone, a hydroxamic acid, a thiol, or a phosphoramide. In particular aspects, the urease inhibitor comprises agrotain or acetohydroxamic acid. In other aspects, the urease inhibitor comprises a combination of more than one of the above-mentioned urease inhibitors.

In some aspects, systems and compositions comprising: milk, Streptococcus thermophilus bacteria, and a urease inhibitor are provided. In further aspects, systems and compositions comprising: milk, Streptococcus thermophilus bacteria, Lactococcus bacteria and a urease inhibitor are provided.

In yet another aspect, systems and compositions comprising cheese and a urease inhibitor are provided.

In certain aspects, methods of producing reduced open-texture cheese comprising:

a) contacting milk with: i) urease positive Streptococcus thermophilus bacteria and a urease inhibitor, and/or ii) urease negative Streptococcus thermophilus bacteria, which are not able to release ammonia from urea at same level as wild-type bacteria; and b) fermenting the milk under conditions such that initial cheese is produced; and c) aging the initial cheese for a period of time such that reduced-texture cheese is produced which has a reduced amount of open-texture compared to control cheese, wherein the control cheese is produced in the same manner as the reduced open-texture cheese but employs the urease positive Streptococcus thermophilus bacteria without the urease inhibitor are provided.

In some aspects, the period of time for the aging is at least 1 month (e.g., at least 1 month . . . 2 months . . . 3.5 months . . . 5 months . . . 6 months . . . 12 months . . . 2 years . . . or longer). In other aspects, the reduced-texture cheese is a gassy cheese. In some aspects, the reduced-texture cheese is a hard or semi hard cheese, for example Cheddar, Red Leicester, American cheese, gouda, edam, emmental, an Italian cheese such as Parmesan, Parmigiano, Regiano, Grana Padano, Provolone, Pecorino, or Romano, or any variety of blue cheese. In further aspects, the reduced-texture cheese is cheddar cheese. The expression “open-texture” can include slits, cracks, eyes, holes, fractures, and combinations thereof. In particular aspects, the reduced open-texture cheese contains no, or essentially no, visible slits, cracks, fractures and the like. In other aspects, the reduced open-texture cheese contains at least 10% less open texture than said control cheese after period of time (e.g., at least 10% . . . 25% . . . 40% . . . 65% . . . 75% . . . 85% . . . 95% . . . or 99% less open texture than the control cheese after a period of time, such as 1 month . . . 6 months . . . 2 years . . . etc).

In other aspects, compositions comprising a cheese selected from the group consisting of: cheddar, Red Leicester, American cheese, gouda, edam, emmental, an Italian cheese like Parmesan, Parmigiano, Regiano, Grana Padano, Provolone, Pecorino, and Romano, or any variety of blue cheese, and a urease inhibitor are provided. In additional aspects, the cheese contains no, or essentially no, visible slits, cracks, fractures and the like.

### DETAILED DESCRIPTION

One of the problems with the use of S. thermophilus for making cottage cheese is that the cheese curds float to the top of the vat, which is undesirable. Due to the floating curds, the cheese is very difficult to process the vat. Without wishing to be bound by theory, it is believed that the floating cheese curd problem in cottage cheese production is due to urease activity associated with S. thermophilus. As such, in some aspects, methods and compositions for making cottage cheese that employ a urease inhibitor and/or S. thermophilus bacteria that do not produce active urease enzymes, or that produce a lower quantity of urease enzymes than wild-type S. thermophilus bacteria, or that produce urease enzymes that have less activity than those produced by wild-type S. thermophilus bacteria, are provided.

The carbon dioxide released by urease enzymes is also believed to be a cause of the floating curd problem. The inventor has recognized that, when S. thermophilus ur(+) bacteria are used, the presence of floating curd depends on the urea levels of the milk that is used. Also, the amount of floating curd (measured in curd height), may be from about 10 cm to about 20 cm when S. thermophilus ur(+) bacteria are used. What is more, the levels of floating curd increase when the temperature is increased, such as during a cooking step. This observation is consistent with the presence of carbon dioxide gas trapped in the curd. The volume of carbon dioxide trapped in the curd increases with increasing temperature. As the volume of trapped carbon dioxide increases, the buoyancy of the curd also increases. As the curd becomes more buoyant, more curd will float.

When S. thermophilus ur(−) bacteria are used, however, the amount of floating curd is reduced or eliminated. Without wishing to be bound by theory, the absence of urease enzymes is believed to correspond to an absence of produced carbon dioxide because urea is not hydrolyzed into ammonium and carbon dioxide. Without the production of carbon dioxide by bacteria, the curd does not become buoyant, reducing or eliminating floating curd.

U.S. Pat. No. 6,962,721 discloses a S. thermophilus that is partially or completely deficient in its ability to release ammonia from urea. This patent also explains how to make such S. thermophilus ur(−) bacteria. A person of ordinary skill in the art also knows how to identify whether a particular S. thermophilus strain is a ur(−) strain. For example, a suitable plate assay to test for urease activity is provided in Example 1 of U.S. Pat. No. 6,962,721, which is hereby incorporated by reference in its entirety.

Similarly, without wishing to be bound by theory, one of the problems with the use of S. thermophilus in making hard or semi-hard cheeses, such as and without limitation cheddar and the other hard or semi-hard cheeses mentioned herein, is the presence of open texture in the final cheese product. Without wishing to be bound by theory, it is believed that S. thermophilus ur(+) bacteria are responsible for open-texture such as slits, eyes, cracks, holes, fractures or combinations thereof. The urease produced by S. thermophilus ur(+) bacteria is believed to hydrolyze urea into carbon dioxide and ammonium. At the relevant temperatures, carbon dioxide is a gas. The gaseous carbon dioxide is believed to form the open texture, such as holes, slits, eyes, fractures, cracks, etc.

In one aspect, methods of using urease inhibitors with S. thermophilus (for example, a wild-type S. thermophilus that is able to make active urease) to make any type of cheese are provided. Exemplary cheeses include, but are not limited to, American cheese, Bergenost, Brick cheese, Cottage cheese, Colby cheese, Colby-Jack cheese, Cream cheese, Cup Cheese, Farmer cheese, Liederkranz cheese, Maytag (Blue cheese), Monterey Jack, Muenster cheese, Pepper jack cheese, Pinconning cheese, Provel cheese, String cheese, Swiss cheese, Teleme cheese, Camembert, Brie de Meaux, Roquefort, Boursin, Reblochon, Munster, Pont l'Eveque, Epoisses, Chevre, emmental, any variety of blue cheese, and Tomme de Savoie.

The amount of the urease inhibitor required per vat during manufacturing can be calculated by methods known in the art, for example, using the TOCRIS BIOSCIENCE molarity triangle. Alternatively or in addition, empirical methods can be used to determine the optimized amount to use. In particular aspects, any appropriate amount of urease inhibitor may be used. In certain aspects, appropriate amounts of urease inhibitor are amounts that yield cheese having the desired texture, moisture level, ripening properties, or a combination thereof.

It is contemplated that the urease activity of Streptococcus thermophilus strains is responsible for the open texture (such as cracks, slits, holes, and the like) in gassy cheese such as cheddar. Using Streptococcus thermophilus ur(−), Streptococcus thermophilus ur(+) with an urease inhibitor, or a combination thereof, may, in some aspects, prevent unwanted open texture.

Without wishing to be bound by theory, it is believed that during production of a gassy cheese (for example, hard cheese, semi hard cheese, and the like), such as cheddar, using Lactococci and Streptococcus thermophilus, urea is trapped in the cheese curd. As such, during storage the urea is slowly metabolized by active urease enzymes in Streptococcus thermophilus to form ammonia and carbon dioxide. If the carbon dioxide cannot escape, it may result in unwanted open texture, such as cracks, splits, fractures, and the like, that may be observable, for example, by visual inspection of the cheese. Such formation of open texture may occur, for example, after about 3-4 months of ripening. In some cases, for example where the ripening temperature is increased to 12° C., the open texture is visible even sooner. Without wishing to be bound by theory, it is believed that the urease enzymes are more active at elevated temperatures, but have lower activity at standard ripening temperatures, which in some aspects is 4° C. Additional compositions and ripening conditions according to the aspects described herein will be apparent to those skilled in the art without departing from the scope and spirit of the description herein, which is intended to encompass at least the full scope of the appended claims.

In certain aspects, the amount of time to reach a desired pH using certain ur(−) S. thermophilus bacteria can be decreased, for example, by adding Lactococci bacteria to the milk used in the fermentation process. When one or more strains of Lactococci bacteria are added to the milk, they can optionally be first mixed together with the ur(−) S. thermophilus bacteria. Thus, in one aspect, a composition may comprise bacterial strains, and the bacterial strains may comprise one or more ur(−) S. thermophilus strains and one or more Lactococci bacterial strains. In another aspect, the composition may comprise bacterial strains, and the bacterial strains may consist essentially of one or more ur(−) S. thermophilus strains and one or more Lactococci bacterial strains. In yet another aspect, the composition may comprise bacterial strains, and the bacterial strains may consist of one or more ur(−) S. thermophilus strains and one or more Lactococci bacterial strains

In particular aspects, the amount of time to reach a desired pH can be decreased by adding a formate source. A formate source is a chemical compound or combination of chemical compounds that provide formate, either as the formate anion, protonated formate, or a mixture thereof, when added to milk or fermented milk (e.g. formic acid, salts of formic acid, precursors of formic acid). Formate sources also include organic molecules (e.g. formic anydride), and formic acid salts (e.g. formate, potassium formate, calcium formate, magnesium formate, lithium formate, etc). In some aspects, formic acid or a salt thereof, for example sodium formate, is used with S. thermophilus ur(−) or ur(+) bacteria, for example, with mixtures of one or more ur(−) S. thermophilus strains and one or more lactococci bacterial strains discussed above. In other aspects, an ammonium source, for example ammonium phosphate, is used with ur(−) S. thermophilus bacteria or ur(+) S. thermophilus bacteria, for example, with mixtures of one or more ur(−) S. thermophilus strains and one or more Lactococci bacterial strains discussed above. In particular aspects, both a formate source and an ammonium source are used with S. thermophilus ur(−) or ur(+) bacteria, for example, with mixtures of one or more ur(−) S. thermophilus strains and one or more Lactococci bacterial strains discussed above.

Furthermore, the inventor has shown that a mixture of Streptococcus thermophilus ur(−) bacteria with formate and Lactococci bacteria is just as active as a mixture of Streptococcus thermophilus ur(+) bacteria with formate and Lactococci bacteria. Without wishing to be bound by theory, it is believed that Lactococci bacteria generate other nitrogen containing nutrients that are usable by the Streptococcus thermophilus ur(−) bacteria. These nutrients are believed to be peptides or amino-acids, which are generated by proteolytic enzymes in Lactococci.

### Example 1

Samples of fresh 1% fat milk were treated with various combinations of S. thermophilus bacteria and Lactococci bacteria as shown in Table 1. In each experiment, an acidification curve was determined by measuring the pH of the milk from the time of addition until 250 minutes after addition. Milk from one source was used as the starting material for each experiment. The temperature of the milk was held at 35° C. for the duration of each experiment. The activity of the S. thermophilus bacteria was correlated to the amount of time that it takes for the pH of the milk to reach a particular level.

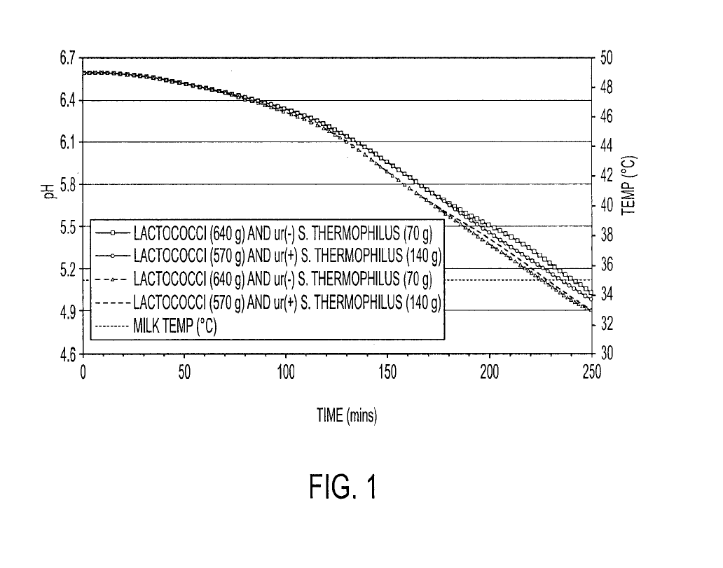

Table 1 and FIG. 1 show exemplary results of four experiments. Mixtures of Lactococci bacteria, formate and either ur(+) or ur(−) S. thermophilus bacteria were added to 1% fat milk, as shown in Table 1. An exemplary acidity profile was determined, and is depicted in FIG. 1. Table 1 also shows selected exemplary data from the acidity profile of FIG. 1 (all time values in Table 1 are in minutes, and the mass of all bacteria is per 1,000 gallons of milk).

TABLE 1

| | Urease | | | | | |
|---|---|---|---|---|---|---|
| | activity of the | | | | | |
| | S. thermophilus | | | | V6-5 (pH/min | |
| | strain | Ta | T5.20 | M6-5 | 10\hat{ }4) | T4.64 |
| Lactococci (570 g), S. thermophilus | Ur(+) | 54.96818 | 221.9714 | −0.00962 | −9.62 | 293.2863 |
| (140 g) | Ur(−) | 53.4535 | 218.2618 | −0.010105 | −10.105 | 294.7521 |
| and sodium formate (10 ppm) | | | | | | |
| Lactococci (640 g), S. thermophilus | Ur(+) | 52.395 | 233.6455 | −0.009025 | −9.025 | 311.678 |
| (70 g) and | Ur(−) | 54.34780 | 227.5821 | −0.00979 | −9.79 | 306.3275 |
| sodium formate (10 ppm) | | | | | | |

FIG. 1 and Table 1 show that when 70 g S. thermophilus is used instead of 140 g S. thermophilus, the milk takes a longer time to reach pH 5.2 (T5.2) and pH 4.65 (T4.65). This is so even though the time before the acidification starts (Ta) is approximately the same for all strains. Nonetheless, the slope of the line from pH 6 to pH 5 (M6-5) and the “velocity” of S. thermophilus action in pH units per minute between pH 6 and pH 5 (V6-5) is similar (within the experimental error) for all four experiments. Further, for experiments using the same amount of S. thermophilus the acidification curves when ur(−) S. thermophilus is used in place of ur(+) S. thermophilu are similar. Thus, the exemplary results in FIG. 1 demonstrate that ur(−) S. thermophilus with Lactococci and formate act as rapidly as ur(+) S. thermophilus with Lactococci and formate.

Thus, one aspect relates to increasing the rate of action of a ur(−) S. thermophilus bacteria on milk by adding one or more Lactococci bacteria to the milk with the S. thermophilus bacteria. In one aspect, the one or more Lactococci bacteria are added at any appropriate time. In another aspect, the Lactococci bacteria are added at the same time as the S. thermophilus bacteria.

### Example 2

Samples of 2% fat milk taken from the same batch were treated with ur(+) S. thermophilus without formate, ur(−) S. thermophilus without formate, and ur(−) S. thermophilus with 10 ppm sodium formate. Lactococci bacteria were not used. The milk was maintained at 40° C., and pH measurements were taken for 350 minutes.

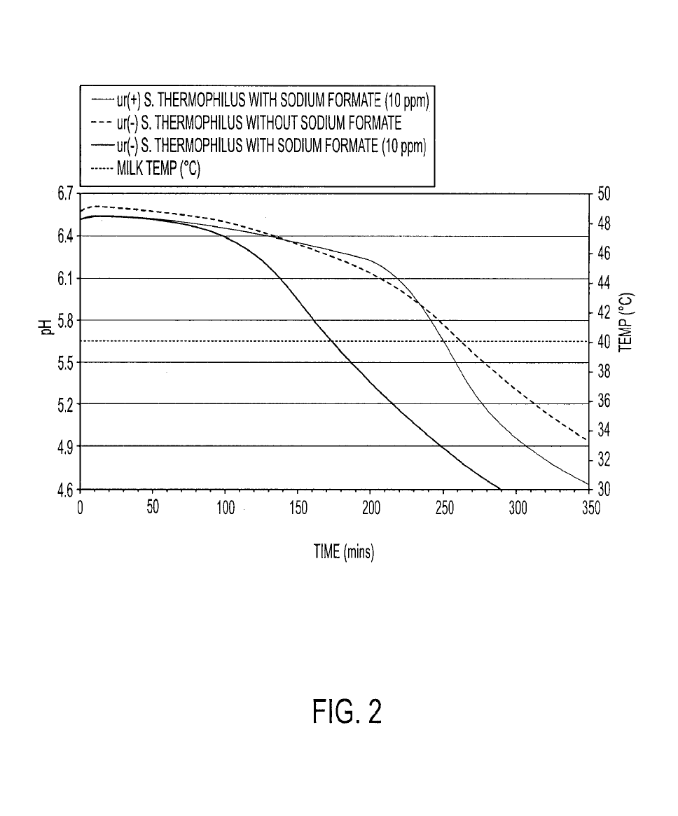

FIG. 2 is a graph showing exemplary results from this experiment. Upon addition of 10 ppm sodium formate, the rate of pH decrease observed after addition of the ur(−) S. thermophilus in combination with formate is accelerated compared to the rate of pH decrease when ur(−) S. thermophilus is added without formate. The rate of pH decrease of ur(−) S. thermophilus in combination with formate is similar to the rate of pH decrease for ur(+) S. thermophilus.

Thus, in one particular aspect, the rate of action of S. thermophilu on milk is increased by adding formic acid or a formate.

### Example 3

Four experiments were conducted in which milk was treated with various bacteria. In experiment 1, only Lactococci bacteria were added. In experiment 2, a blend of Lactococci bacteria and ur(+) S. thermophilus bacteria were added. In experiment 3, a blend of Lactococci bacteria, ur(+) S. thermophilus bacteria, and the urease inhibitor flurofamide were added. In experiment 4, only ur(+) S. thermophilus bacteria were added.

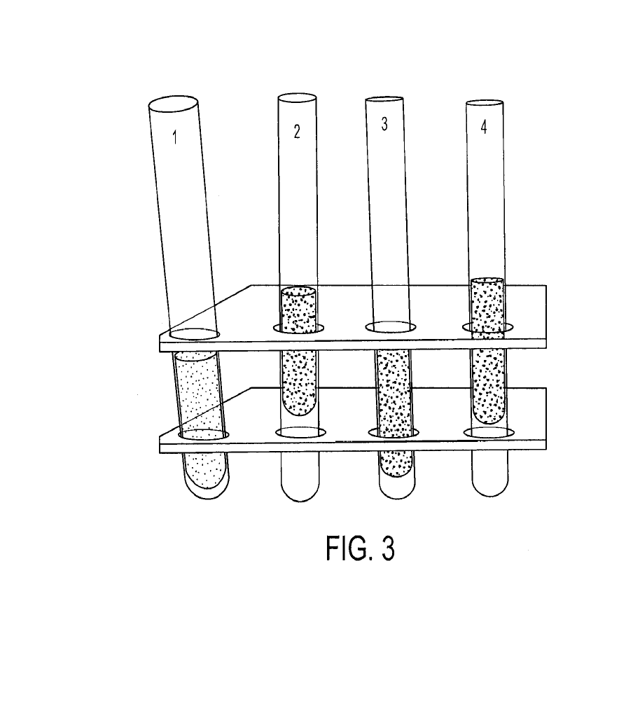

In each experiment, the milk was fermented with the bacteria at 35° C. until the cheese reached a pH of 4.65. A sample of the cheese was placed into a test tube, which was heated at about 66° C. for about 10 minutes. After 10 minutes of heating, a small pipette or thin wire was used to agitate the sample. The samples were held at about 66° C. for another ten minutes, at which time the photograph of the test tubes depicted in FIG. 3, was taken.

FIG. 3 shows that there is no floating curd in test tubes 1 and 3, which correspond to experiments 1 and 3, respectively. Test tubes 2 and 4, which correspond to experiments 2 and 4, respectively, contain floating curd. These results illustrate the effect of urease on the amount of floating curd resulting from fermentation. Test tube 1, a negative control that contains only Lactococci, shows no floating curd because Lactococci do not contain urease enzymes that can hydrolyze urea in milk. Test tube 4 is a positive control that contains ur(+) S. thermophilus, which has urease enzymes that can hydrolyze urea in milk. Test tube 2, which contains floating curd, and test tube 3, which does not, both contain a mixture of Lactococci and S. thermophilus. These test tubes differ only in that test tube 3, which does not contain floating curd, was made in the presence of a urease inhibitor that inactivates the urease enzyme and prevents it from hydrolyzing urea to carbon dioxide and ammonia. Thus, test tubes 2 and 4, which include an active urease enzyme, exhibit floating curd, whereas test tubes 1 and 4, which either have no urease enzyme (test tube 1) or have a urease enzyme that is deactivated by an inhibitor (test tube 4) do not contain floating curd.

### Example 4

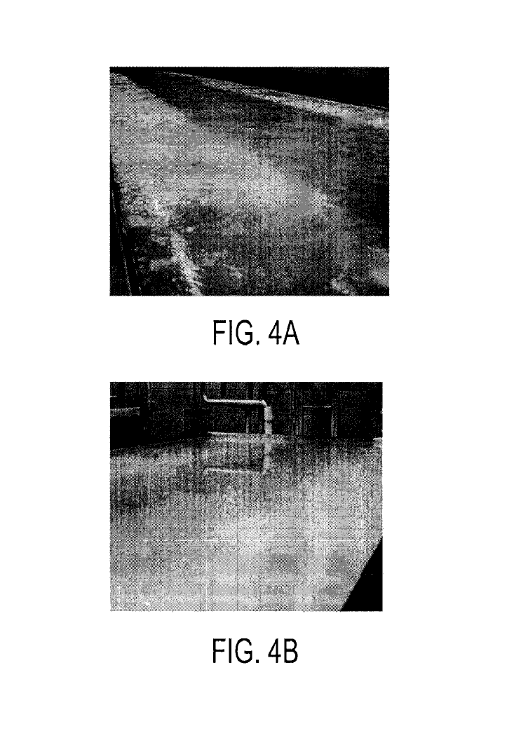

Two vats of milk were inoculated with S. thermophilus and Lactococci bacteria. The first vat was inoculated with a ur(+) strain of S. thermophilus, and the second with a ur(−) strain of S. thermophilus. The two vats were photographed after curds formed. FIG. 4A is a photograph of the first vat. FIG. 4A shows the curds floating on top of the vat. FIG. 4B is a photograph of the second vat. FIG. 4B shows that no curds are floating on top of the milk. The results of this experiment are consistent with the results of Example 3, in that the floating curd only appears when a ur(+) strain of S. thermophilus is used.

In particular aspects, a urease inhibitor may be added to ur(+) bacteria, such as ur(+) S. thermophilus, in order to reduce the amount of floating curd relative to the amount that is present without the urease inhibitor. In certain aspects, the urease inhibitor results in no floating curd. In specific aspects, the amount of floating curd is reduced, relative to the amount that is produced when ur(+) S. thermophilus bacteria is used, by using ur(−) S. thermophilus bacteria. In some aspects, the use of ur(−) S. thermophilus bacteria results in no floating curd.

### Example 5

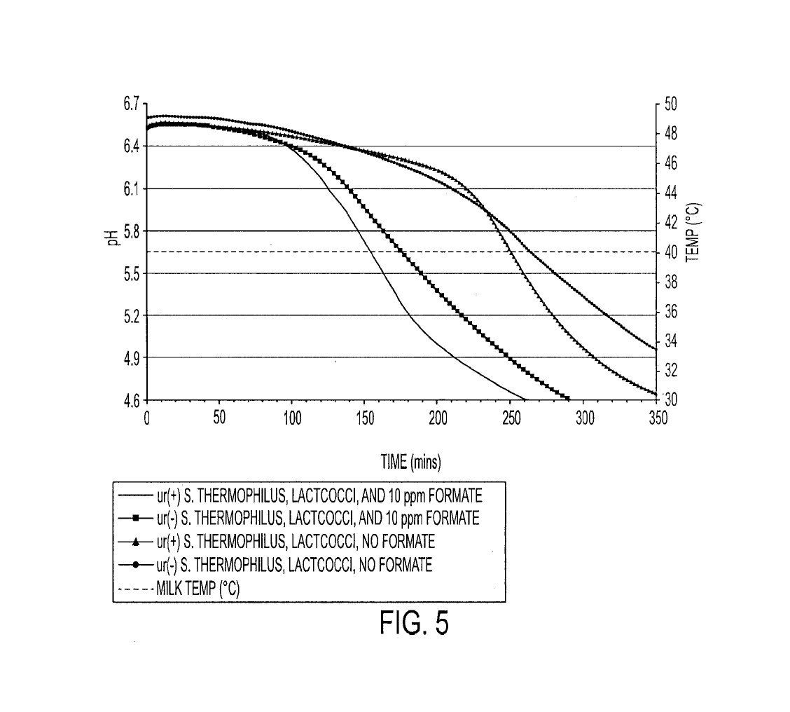

Samples of fresh 1% fat milk were treated with various combinations of S. thermophilus bacteria and formate (in the form of sodium formate) as shown in Table 2. In each experiment, an acidification curve was determined by measuring the pH of the milk from the time of addition until 250 minutes after addition. Milk from one source was used as the starting material for each experiment. The temperature of the milk was held at 35° C. for the duration of each experiment. The activity of the S. thermophilus bacteria was correlated to the amount of time that it takes for the pH of the milk to reach a particular level. An exemplary activity profile was determined, and is depicted in FIG. 5. Selected data from the graph in FIG. 5 also appear in Table 2, which uses the same abbreviations as Table 1, above, and also gives times in minutes.

TABLE 2

| | Ta | T5.20 | M6-5 | T4.64 |
|---|---|---|---|---|
| ur(+) S. thermophilus, | 79.49704 | 180.9662 | −0.01577 | 248.8029 |
| lactoccocci, 10 ppm | | | | |
| formate | | | | |
| ur(−) S. thermophilus, | 80.07301 | 215.9344 | −0.0116 | 280.9252 |
| lactoccocci, 10 ppm | | | | |
| formate | | | | |
| ur(+) S. thermophilus, | 95.01494 | 277.926 | −0.01605 | 345.2202 |
| lactoccocci, no formate | | | | |
| ur(−) S. thermophilus, | 88.57867 | 314.2438 | −0.00905 | 398.4624 |
| lactoccocci, no formate | | | | |

The activity profile depicted in FIG. 5, and the selected data displayed in Table 2 show several surprising and unexpected results. These experiments, along with the experiments described in Example 1, show that the addition of formate can unexpectedly increases the activity of both ur(+) and ur(−) S. thermophilus bacteria, as measured, for example, by the rate of acidification of the milk. Further, whereas ur(−) S. thermophilus strains may be less active than ur(+) strains, addition of formate to the ur(−) strains surprisingly increases their activity, so that ur(−) S. thermophilus with formate is more active than ur(+) S. thermophilus without formate.

Without wishing to be bound by theory, it is believed that the enzyme pyruvate formate lyase, present in S. thermophilus, is anaerobic and has little or no activity in the presence of oxygen. When it is active, pyruvate formate lyase is believed to produce formate. When oxygen is present, S. thermophilus activity is believed to decrease because the amount of formate produced by pyruvate formate lyase is reduced. When an external formate source, such as sodium formate, is added, the activity of S. thermophilus is increased. Formate sources other than sodium formate, such as formic acid, potassium formate, magnesium formate, calcium formate, or any other acceptable formate, may also be used for this purpose.

When S. thermophilus, for example ur(−) S. thermophilus, is used to make certain fermented milk products, such as yoghurt, fermented milk, or hard or semi-hard cheese, for example and without limitation, cheddar, the S. thermophilus may be present in the resulting product, such hard or semi-hard cheese. In the manufacture of such products, such as yoghurt, fermented milk, or hard or semi-hard cheese, the milk may not be heated to a temperature high enough to kill the S. thermophilus bacteria. For example, the cook temperature used during the manufacture of such products, such as yoghurt, fermented milk, or hard or semi-hard cheese, such as cheddar, may be about 101° F. to about 103° F., which may be insufficient to kill one or more S. thermophilus bacteria, such as ur(−)S. thermophilus bacteria. Thus, the resulting fermented milk product may comprise live S. thermophilus bacteria, such as live ur(−) S. thermophilus bacteria. As a non-limiting example, the cell count of S. thermophilus, in a finished hard or semi-hard cheese such as cheddar may be about 10⁸ to about 10⁹ cells per gram of cheese.

On the other hand, when cottage cheese is manufactured with S. thermophilus, for example ur(−) S. thermophilus bacteria, the S. thermophilus bacteria are killed during cooking, at temperatures from about 125° F. to about 150° F., for example from about 136° F. to about 140° F. Without being bound by theory, killing S. thermophilus during the manufacture of cottage cheese is preferred, because cottage cheese contains curd and a dressing, which is believed to contain a significant amount of sugars, for example, lactose and other sugars, which can be further metabolized by S. thermophilus, for example ur(−) S. thermophilus, bacteria. If the S. thermophilus bacteria are not killed during the cooking phase of cottage cheese manufacture, then it is believed that the S. thermophilus bacteria will continue to metabolize the sugar, which will give an unsuitable product, for example, a product that is too acidic.

### Example 6

In an exemplary method of manufacture, milk is added to a vat, such as any appropriate cheese-making vat known in the art. A bacterial culture including ur(−) S. thermophilus and one or more Lactococci strains is added at an appropriate time (e.g., when the vat is about 10% to about 40% full of milk, or about 15% to about 35% full of milk, or about 20% to about 30% full of milk, or 25% full of milk). Formate, such as sodium formate, can optionally be added to the vat at an appropriate time and in an appropriate amount (e.g., about 1 ppm to about 50 ppm, or about 5 ppm to about 20 ppm, or about 10 ppm). The vat is initially set to an appropriate set temperature for making the desired type of cheese, such as cheddar (e.g., about 88 to about 90 degrees F.). The resulting mixture is allowed to set for an appropriate set time (e.g., about 20 to about 25 minutes). During this time, the milk can form curds, which can then be washed and cut, followed by a heal time that can be appropriate for making the desired type of cheese, such as cheddar. The heal time can be, for example, about 10 to about 15 minutes.

After the cut and heal, the temperature of the vat is raised to an appropriate cook temperature (e.g., about 101° to about 103° F.). The vat can be held at the cook temperature for the appropriate amount of time depending on the type of cheese desired. For example, the vats can be held at the cook time for about 20 to about 40 minutes. The content of the vat is set, for example by cooling to an appropriate temperature for an appropriate set time (e.g., about 2.5 to about 2.9 hours). After this set time, the cheese in the vat may be salted with an appropriate amount of salt. The cheese from the two vats can then be allowed to ripen for an appropriate amount of time, depending on the type of cheese desired. The ripening time may be, for example, about 1 to about 60 months, about 6 to 36 months, about 9 to about 24 months, or about 18 months.

The procedure described above can also be repeated using a bacterial culture including ur(+) S. thermophilus and one or more Lactococci. The cheese produced from ur(−) S. thermophilus can then be compared to the cheese produced using ur(+) S. thermophilus. In one aspect, the cheese made with ur(−) S. thermophilus has fewer slits, fractures, eyes, holes, cracks, and/or other open texture when compared with the cheese made from ur(+) S. thermophilus. For example, the cheese made from ur(−) S. thermophilus has essentially no visible slits, cracks, or holes when viewed with a human eye.

Thus, based on the description provided herein, a person of ordinary skill in the art can manufacture, among other things, a fermented milk product comprising one or more ur(−) S. thermophilus bacteria. A hard or semi-hard cheese, for example and without limitation, cheddar cheese, may contain one or more ur(−) S. thermophilus bacteria. The ur(−) S. thermophilus bacteria may be any strain of ur(−)S. thermophilus bacteria, such as the strains mentioned above. The cell count of the ur(−)S. thermophilus bacteria in the cheese may be from about 10⁸ to about 10⁹ cells per gram of cheese.

Thus, a fermented milk product may comprise one or more live ur(−) S. thermophilus bacteria. The product may be a hard or semi-hard cheese. This fermented milk product containing one or more live ur(−) S. thermophilus bacteria may be an ingredient for a consumable, such as a nutraceutical, pharmaceutical, food additive, or baby formula.

Similarly, a method of making a fermented milk product may involve contacting milk with one or more ur(−) S. thermophilus bacteria. The milk may also be contacted with one or more Lactococci bacteria. A fermented milk product, such as fermented milk, yoghurt, hard or semi-hard cheese, etc. may be manufactured by this method.

In some aspects, the one or more ur(−) S. thermophilus bacteria may be added as part of a direct vat culture, which may also include one or more Lactococci bacteria. In particular aspects the direct vat culture may also include formate. In certain aspects, a direct vat culture comprises one or more ur(−) S. thermophilus bacteria. In other aspects, a direct vat culture comprises one or more ur(−) S. thermophilus bacteria and one or more Lactococcus bacteria. In still other aspects, a direct vat culture comprises one or more ur(−) S. thermophilus bacteria, one or more Lactococcus bacteria, and at least one formate source. In yet other aspects, a direct vat culture consists essentially of, or consists of, one or more ur(−) S. thermophilus bacteria and one or more Lactococcus bacteria. In further aspects, a direct vat culture consists essentially of, or consists of, one or more ur(−) S. thermophilus bacteria, one or more Lactococcus bacteria, and one or more formate source. The formate source may be formic acid, sodium formate, potassium formate, magnesium formate, calcium formate, or other acceptable formate sources.

In certain aspects, the one or more ur(−) S. thermophilus bacteria may be added as part of a bulk starter, which may also include one or more Lactococci bacteria. In particular aspects the bulk starter may also include formate. In certain aspects, a bulk starter comprises one or more ur(−) S. thermophilus bacteria. In other aspects, a bulk starter comprises one or more ur(−) S. thermophilus bacteria and one or more Lactococcus bacteria. In still other aspects, a bulk starter comprises one or more ur(−) S. thermophilus bacteria, one or more Lactococcus bacteria, and at least one formate source. In yet other aspects, a bulk starter consists essentially of, or consists of, one or more ur(−) S. thermophilus bacteria and one or more Lactococcus bacteria. In further aspects, a bulk starter consists essentially of, or consists of, one or more ur(−) S. thermophilus bacteria, one or more Lactococcus bacteria, and one or more formate source. The formate source may be formic acid, sodium formate, potassium formate, magnesium formate, calcium formate, or other acceptable formate sources.

Although the description herein is in connection with specific preferred aspects, it should be understood that the claims should not be unduly limited to such specific aspects. For example, while particular strains of S. thermophilus ur(−) bacteria and particular types of milk and cheese are used to illustrate the basic principles described herein and means for practicing the associated methods, the artisan would readily understand that the same results could be obtained with other strains of S. thermophilus ur(−) bacteria, could be applied to other types of milk, and could be used to make other types of fermented milk products or cheeses. Indeed, various modifications of the described modes for carrying out the aspects described herein that are obvious to those skilled in the relevant fields are intended to be within the scope of the following claims.

1-71. (canceled)

72. A direct to vat culture comprising at least one ur(−) S. thermophilus bacteria, at least one Lactococcus bacteria and at least one formate source.

73. The direct vat culture of claim 72, wherein the at least one formate source comprises sodium formate.

74. The direct vat culture of claim 72, consisting essentially of at least one ur(−) S. thermophilus bacteria, at least one Lactococcus bacteria, and at least one formate source.

75. A bulk starter comprising at least one ur(−) S. thermophilus bacteria, at least one Lactococcus bacteria and at least one formate source.

76. The bulk starter of claim 75, wherein the at least one formate source comprises sodium formate.

77. The bulk starter of claim 75, consisting essentially of at least one ur(−) S. thermophilus bacteria, at least one Lactococcus bacteria, and at least one formate source.

78. A method of accelerating the acidification of milk, comprising:
contacting milk with at least one S. thermophilus bacteria, wherein the at least one S. thermophilus bacteria comprises ur(−) S. thermophilus bacteria;
contacting the milk with at least one Lactococcus bacteria; and
contacting the milk with at least one formate source.

79. The method of claim 78, wherein formate source is sodium formate.

80. The method of claim 78, wherein the formate source is potassium formate

81. The method of claim 78, wherein the concentration of the formic acid or salt thereof is about 1 ppm to about 20 ppm.

82. The method of claim 78, wherein the formate is in a concentration of about 10 ppm.

83. The method of claim 78, wherein the at least one S. thermophilus bacteria consists essentially of ur(−) S. thermophilus bacteria.

84. The method of claim 78, wherein the at least one Lactococcus bacteria comprises at least one of L. cremoris and L. lactis.

85. A method of manufacturing a hard or semi-hard cheese comprising contacting milk with a ur(−) S. thermophilus bacteria, with at least one formate source and milk with at least one Lactococci bacteria, and forming the hard or semi-hard cheese wherein essentially no open texture is detectable by visual inspection of the hard or semi-hard cheese.

86. The method of claim 85, wherein the hard or semi-hard cheese is selected from the group consisting of cheddar, red Leicester, American, gouda, edam, emmental, Parmesan, Parmigiano, Regiano, Grana Padano, Provolone, Pecorino, Romano, mozzarella, blue cheeses, and pizza cheese.

87. The method of claim 85, wherein the hard or semi-hard cheese is cheddar.

88. The method of claim 85, wherein the at least one formate source is sodium formate.

89. The method of claim 85, wherein the at least one formate source is potassium formate.

90. The method of claim 85, wherein the at least one formate source is in a concentration of about 1 ppm to about 20 ppm.

91. The method of claim 85, wherein the formate is in a concentration of about 10 ppm.

92. The method of claim 85, further comprising the step of forming curd, wherein essentially none of the curd floats on the milk.

93. The method of claim 85, wherein the Lactococci bacteria comprise at least one of Lactococcus cremoris and Lactococcus lactis.

94. A hard or semi-hard cheese made by the process of claim 85.

95. The hard or semi-hard cheese of claim 94, wherein the hard or semi-hard cheese is selected from the group consisting of cheddar, red Leicester, American, gouda, edam, emmental, Parmesan, Parmigiano, Regiano, Grana Padano, Provolone, Pecorino, Romano, mozzarella, blue cheeses, and pizza cheese.

96. The hard or semi-hard cheese of claim 94, wherein the hard or semi-hard cheese is selected from the group consisting of cheddar, red Leicester, American, gouda, edam, emmental, Parmesan, Parmigiano, Regiano, Grana Padano, Provolone, Pecorino, Romano, mozzarella, blue cheeses, and pizza cheese.

97. The hard or semi hard cheese of claim 94, wherein essentially no open texture is detectable by visual inspection of the hard or semi-hard cheese.

98. The hard or semi-hard cheese of claim 94 comprising one or more live ur(−) S. thermophilus bacteria.

99. The hard or semi-hard cheese of claim 94, wherein the one or more live ur(−) S. thermophilus bacteria is selected from the group consisting of CNCM I-2980, DSM21892, CNCM I-3617, CNCM I-3617, CHCC4325, DSM18344, DSM1811, and combinations thereof.
